(19) United States
(12) Patent Application Publication
(10) Pub. No.: US 2012/0015010 A1
(43) Pub. Date: Jan. 19, 2012

(54) Compositions Comprising Pigment Particles Entrapped In Collapsed Polymeric Microspheres, And Methods Of Making The Same

(76) Inventors: PHILLIP CUMMINS, LIVINGSTON, NJ (US); MILAN FRANZ SOJKA, CORAM, NY (US); RACHEL J. CULHANE, COMMACK, NY (US); CHRISTINA G. FTHENAKIS, DIX HILLS, NY (US); JENNIFER L. OKO, SELDEN, NY (US); JOHN DUDLEY DREHER, SAYVILLE, NY (US); LINDA J. NAJDEK, EAST ISLIP, NY (US); ISAAC D. COHEN, BROOKLYN, NY (US)

(21) Appl. No.: 12/987,278

(22) Filed: Jan. 10, 2011

Related U.S. Application Data
(60) Provisional application No. 61/293,753, filed on Jan. 11, 2010.

(51) Int. Cl. A61K 8/02; A61K 9/14; B01J 13/22

(52) U.S. Cl. 424/401; 427/213.31; 424/497

(57) ABSTRACT

Topical compositions containing pigment particles entrapped in microspheres are provided. Each of the microspheres contains a collapsed polymeric shell that has entrapped therein one or more pigment particles. The microsphere-entrapped pigment particles are characterized by enhanced color intensity, improved stability, and better dispersibility, which can readily be used either alone or in combination with other skin active ingredients to form better colored cosmetics.

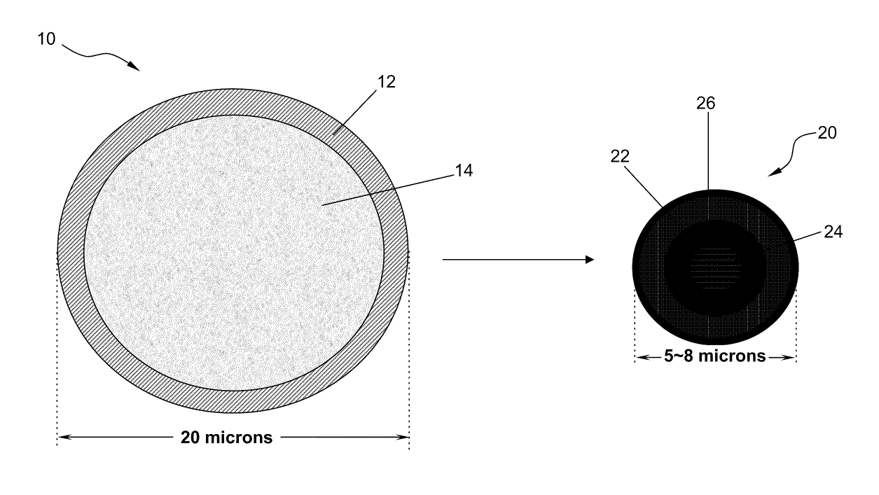

### CROSS-REFERENCE TO RELATED APPLICATIONS

The present application claims priority from U.S. Provisional Patent Application Ser. No. 61/293,753, filed Jan. 11, 2010.

### FIELD OF THE INVENTION

The present invention relates to topical compositions comprising pigment particles with improved dispersibility, reduced tendency to agglomerate, and enhanced color intensity, as well as methods of making the same.

### BACKGROUND OF THE INVENTION

Cosmetic or topical compositions typically comprise one or more inorganic or organic pigment particles, such as metal oxides, lakes, Red 6, Red 21, Brown, Russet and Sienna dyes. Such pigment particles are often insoluble in the respective solvent or carrier system and if so remain dispersed or suspended in the cosmetic or topical compositions.

However, whenever there are changes in the pH and temperature in the surrounding environment, the dispersed or suspended pigment particles may agglomerate with one another and precipitate out of the composition. Further, the smaller the particle size, the larger the active surface area, and the more susceptible such pigment particles are toward adverse interactions or interference with other ingredients or components in the cosmetic or topical compositions, which may destabilize the cosmetic or topical compositions or reduce the overall performance thereof.

There is therefore a continuing need for treating or modifying the pigment particles of cosmetic or topical compositions in order to eliminate or mitigate the above-described drawbacks and improve the overall stability of the compositions without adversely affecting the chemical and physical properties of the pigment particles.

There is also a need for improving the chemical and/or physical properties of the pigment particles through surface treatment or modification.

### SUMMARY OF THE INVENTION

In one aspect, the present invention relates to a topical composition comprising a dispersion of microspheres in a cosmetically or pharmaceutically acceptable carrier, wherein each of the microspheres comprises a collapsed polymeric shell having entrapped therein one or more pigment particles.

In another aspect, the present invention relates to a microsphere comprising a collapsed polymeric shell having entrapped therein one or more pigment particles, while the collapsed polymeric shell is further coated with a liquid-impermeable membrane.

In a still further aspect, the present invention relates to a method for treating pigment particles, comprising:
(a) forming a gelled mixture by mixing either simultaneously or sequentially in any order: (1) hollow microspheres each comprising a deformable polymeric shell having entrapped therein an expandable fluid, (2) a polar organic solvent capable of swelling but not dissolving the polymeric shells of the hollow microspheres, and (3) pigment particles, wherein micro-channels are formed in the swelled polymer shells to allow entry of the pigment particles into the hollow microspheres and exit of the expandable fluid therefrom, thereby forming microspheres that each comprises a collapsed polymeric shell in a gelled state and has one or more of said pigment particles entrapped therein;
(b) removing the expandable fluid and the polar organic solvent from the gelled mixture; and
(c) coating the microspheres with a film-forming material to form a liquid-impermeable membrane thereon.

Other aspects and objectives of the present invention will become more apparent from the ensuing description, examples, and claims.

### BRIEF DESCRIPTION OF THE DRAWINGS

The figure illustratively shows schematic views of: (1) an untreated hollow microsphere with a deformable polymeric shell and an expandable fluid entrapped therein, and (2) a microsphere containing a collapsed polymeric shell with pigment particles entrapped therein and a liquid-impermeable membrane coated thereover, which is formed by processing the untreated hollow microsphere according to one embodiment of the present invention.

### DETAILED DESCRIPTION OF THE INVENTION, AND PREFERRED EMBODIMENTS THEREOF

The present invention provides modified pigment particles that are useful in cosmetic or topical compositions, as well as methods for modifying pigment particles. Specifically, the pigment particles are entrapped in polymeric microspheres having an average particle size that is at least 10 times, preferably 20 times, more preferably 50 times, and most preferably 100 times, larger than the average particle size of the pigment particles themselves. Each of the microspheres comprises a collapsed polymeric shell having entrapped therein one or more pigment particles.

It has been discovered by the inventors that when entrapped into the polymeric microspheres, the color intensity of the pigment particles is significantly enhanced in comparison with un-treated pigment particles. Therefore, the microsphere-entrapped pigment particles of the present invention can be used to form cosmetic products of more vibrant colors, or pigment particles of lesser amount can be used in a cosmetic formulation to achieve the same coloring effect. Further, the microsphere-entrapped pigment particles the significantly larger microspheres provide improved structural and spatial stability and allow formation of cosmetic products with extended shelf life.

Entrapment of the pigment particles is achieved in the present invention by first providing hollow microspheres with deformable polymeric shells having encapsulated therein an expandable fluid, which are then mixed with, either sequentially in any order or simultaneously, a polar organic solvent capable of swelling but not dissolving the polymeric shells of the hollow microspheres and solid particles to be entrapped. A gelled mixture is thereby formed, which contains microspheres with polymeric shells in a gelled state, which are sufficiently swelled so as to have micro-channels or through-holes formed therein to allow entry of the solid particles into the microspheres. Such micro-channels or through-holes in the swelled polymeric shells of the microspheres also allow exit of the expandable fluid from the microspheres, thereby causing immediate collapse or implosion of the polymeric shells and entrapping the solid particles inside the microspheres. Subsequently, the expandable fluid and the polar organic solvent are removed from the gelled mixture. Preferably but not necessarily, a film-forming material is coated over the collapsed polymeric shells to form a liquid-impermeable membrane thereon, which functions to isolate the collapsed polymeric shells of the microspheres from any solvent in the surrounding environment that may swell or otherwise affect the structural integrity of such polymeric shells. In this manner, the solid particles can be securely entrapped inside the microspheres with little or no risk of leaking out.

The hollow microspheres as initially provided (i.e., before mixing with the solid particles and the polar organic solvent) are preferably expandable hollow polymeric microspheres, each of which contains a deformable polymeric shell that is gas-tight and has enclosed or encapsulated therein an expandable fluid. Upon heating, the enclosed or encapsulated fluid can expand volumetrically to apply pressure on the interior wall of the deformable polymeric shell. At the same time, the elevated temperature may cause the polymeric shell to soften, thereby allowing the entire microsphere to expand in a manner similar to a balloon.

The deformable polymeric shells of the hollow microspheres can be formed of any synthetic or natural crosslinked or un-crosslinked polymer. If the polymer is crosslinked, it is preferred that it is weakly crosslinked. Preferably, but not necessarily, the polymeric shells of the hollow microspheres comprise at least one synthetic polymer obtained by polymerization of one or more ethylenically unsaturated monomers to form homopolymers or copolymers of ethylenically unsaturated monomers or copolymers of ethylenically unsaturated monomers and one or more organic groups. Examples of ethylenically unsaturated monomers that may be suitable include, for example, vinylidene chloride, vinyl chloride, acrylonitrile, acrylic acid and its corresponding C₁-C₂₀ aliphatic or aromatic esters, methacrylic acid and its corresponding C₁-C₂₀ aliphatic or aromatic esters, acrylamide, methacrylamide, vinyl pyrrolidone, alkenes such as styrene, ethylene, propylene, butylene, methylpentene, 1,3-butadiene, and the like. The polymeric shells of the hollow microspheres may also be formed of suitable synthetic polymers, such as polyesters, polyamides, polyphthalamides, polyimides, polycarbonates, polyketones, cellulose acetate, polysulfones, polyphenylene sulfides, polyphenylene oxides, polylactic acids, polyvinylpyrrolidone, polystyrene, polyacrylonitrile, polyacrylamide, polymethylmethacrylate, polyacrylates, and copolymers of the above-listed polymers. In a particularly preferred embodiment, the deformable polymeric shells of the hollow microspheres are formed of a copolymer of vinylidene chloride, acrylonitrile, and/or methyl methyacrylate.

The expandable fluid inside the hollow microspheres of the present invention can be any suitable gas (e.g., air or nitrogen) or volatile liquid hydrocarbons (e.g., isobutane or isopentane). Preferably, the expandable fluid is selected from the group consisting of air, nitrogen, isobutane, and isopentane. More preferably, the expandable fluid is either isobutane or isopentane.

Hollow microspheres having deformable polymeric shells comprised of a copolymer of vinylidene chloride, acrylonitrile, and methylmethacrylate with an expandable fluid comprised of isobutane or isopentane are commercially available under the trade name of EXPANCEL® from Expancel, Inc. at Duluth, Ga. The EXPANCEL® hollow microspheres are available in various forms, e.g., dry, wet, unexpanded or pre-expanded. Both the dry, unexpanded microspheres (EXPANCEL® DU) and the dry, expanded microspheres (EXPANCEL® DE) can be used in the present invention for entrapping and stabilizing the solid particles. The EXPANCEL® DU microspheres have an average particle size ranging from about 6 to about 40 microns and a density of about 1-1.3 g/cm³. The EXPANCEL® DE microspheres have an average particle size ranging from about 20 to about 150 microns and a density of about 0.03-0.07 g/cm³.

Any suitable polar organic solvent that can sufficiently swell, but not dissolve, the polymeric shells of the hollow microspheres can be used to treat the hollow microspheres described hereinabove. Examples of polar organic solvents that can be used in practice of the present invention include, but are not limited to: dimethylformamide, dimethylchloride, trichloroethylene (TCE), chloroform, methanol, ethanol, isopropanol, acetone, ethyl acetate, butyl acetate, and methyl ethyl ketone (MEK). Acetone is most preferred in the present invention. Upon mixing with untreated hollow microspheres, the polar organic solvent can swell the polymeric shells of the hollow microspheres significantly and thereby convert the gas-tight polymeric shells of the untreated hollow microspheres into a gelled state with multiple micro-channels or pores formed therein.

The pigment particles to be entrapped according to the present invention can be any particulate pigment materials suitable for use in cosmetic and skin care products, including, but not limited to, those listed in the CTFA International Color Handbook, Fourth Edition 2008, as published by the Cosmetics, Toiletry and Fragrances Association, Inc. (CTFA). For example, the pigment particles may comprise one or more selected from: metallic oxides, such as iron oxides of various colors, titanium dioxide, zinc oxide, cerium oxide, zirconium dioxide, chromium oxide, and the like; organic pigments, such as D&C or FD&C colors or organic dyes including Red 6, Red 21, Brown, Russet and Sienna dyes and mixtures thereof; Lakes, such as aluminum Lakes, calcium Lakes and barium Lakes, while more specific examples of which include Red 3 Aluminum Lake, Red 21 Aluminum Lake, Red 27 Aluminum Lake, Red 28 Aluminum Lake, Red 33 Aluminum Lake, Yellow 5 Aluminum Lake, Yellow 6 Aluminum Lake, Yellow 10 Aluminum Lake, Orange 5 Aluminum Lake and Blue 1 Aluminum Lake, Red 6 Barium Lake, Red 7 Calcium Lake, and the like; and any other color-enhancing or color-altering powders, such as talc, mica, kaolin, pearl powder, magnesium carbonate, calcium carbonate, magnesium silicate, aluminum magnesium silicate, silica, silica beads, ultramarine, polyethylene powder, methacrylate powder, polystyrene powder, silk powder, crystalline cellulose, starch, titanated mica, bismuth oxychloride, chromium hydroxide, barium sulfate, polymethylmethacrylates (PMMA), boron nitride, nylon beads, polymeric powders (e.g., BPD 500 powders comprised of hexamethylene diisocyanate/trimethylol hexyllactone crosspolymer and silica that is commercially available from Kobo Products, Inc. at South Plainfield, N.J.), and the like.

To increase the color variety, two or more different types of pigment particles can be combined into one composition. In one specific embodiment of the present invention, such two or more different types of pigment particles are co-encapsulated into the microspheres. In another specific embodiment, such two or more different types of pigment particles are separately encapsulated into microspheres first and then combined together into one formulation.

The average particle size of the pigment particles as used in the present invention should be significantly smaller than that of the hollow microspheres, so that the pigment particles can readily enter and be entrapped by the hollow microspheres. Preferably, the average particle size of the pigment particles is less than 1 micron, more preferably from about 0.001 micron to about 0.1 micron, and most preferably from about 0.01 to about 0.05 micron.

The suggested ranges of such microsphere-entrapped pigment particles are from about 0.1 to 90%, preferably from about 0.5 to 60%, and more preferably from about 1% to about 30% by weight of the total composition. Unless otherwise specified, all the percentages described herein refer to the weight percentage based on the total weight of the final composition.

The hollow microspheres, the polar organic solvent and the pigment particles as described hereinabove are mixed together, either simultaneously or sequentially, to form a gelled mixture. If mixed sequentially, the ingredients can be added and mixed in any suitable order. For example, the hollow microspheres and the pigment particles can be blended together first, followed by addition of the polar organic solvent to form a slurry. For another example, the pigment particles can be dispensed in the polar organic solvent first, and then mixed with the hollow microspheres. For still another example, the hollow microspheres can be added into the polar organic solvent to form a gel first, and the pigment particles are then added into the gel. In any event, all the ingredients are well mixed until a homogenous mixture is formed. The weight ratio between the hollow microspheres and the polar organic solvent is preferably from about 1:3 to about 1:100 and more preferably from about 1:20 to about 1:50, so that the polymeric shells of the hollow microspheres can be sufficiently swelled by the solvent. The weight ratio between the pigment particles and the hollow microspheres can range widely from about 1:10 to about 100:1, preferably from about 2:3 to about 10:1, and more preferably from about 1:1 to about 2:1.

Because the polymeric shells of hollow microspheres are comprised of a non-crosslinked or weakly crosslinked polymer, as mentioned hereinabove, the polar organic solvent molecules, which are sufficiently small in comparison with the polymeric molecules, can enter between the polymeric chains, interrupt the intermolecular bonds between neighboring polymeric chains, and pull the polymeric chains apart from each other. Consequently, the polymeric shells of the hollow microspheres are swelled by the polar organic solvent, so as to form a gelled mixture that contains porous networks of interconnected polymeric chains spanning or dispersed throughout the volume of the polar organic solvent. The polymeric shells of the microspheres in such a gelled state are not longer gas-tight, but have become porous, i.e., with sufficiently large micro-channels therein to allow entry of the pigment particles into the sufficiently swelled microspheres. At the same time, the expandable fluid exit from such microspheres through the micro-channels, causing the gelled polymeric shells to collapse or implode and resulting in shrunk microspheres with significantly decreased overall volume. In this manner, the pigment particles become entrapped within the collapsed polymeric shells of the shrunk microspheres.

Such shrunk microspheres have an average particle size ranging from about 1 to 15 microns, and more from about 5 microns to about 8 microns. The shrunk microspheres are significantly smaller in size than the untreated hollow microspheres. Further, the shrunk microspheres are no longer hollow, but are now filled by the pigment particles with little or no empty space left therein. At the same time, the polymeric shells of the microspheres remain in a gelled state, i.e., swelled by the polar organic solvent. It is important to note that the shrunk microspheres of the present invention, although morphologically and volumetrically modified by the gelling process, remain as separate particles in the gelled mixture with little or no coalescence. Subsequent drying of the gelled mixture therefore forms fine free-flowing powders, which contain microspheres with well-defined surface boundaries and minimum clumping or agglomeration.

The gelling process as described herein is fundamentally different from the well known sol gel process. In a typical sol-gel process, metal alkoxide and metal chloride precursors are first solubilized to form a solution (sol) and then undergo hydrolysis and polycondensation reactions to form a colloid system composed of solid particles dispersed in a solvent, followed by evolvement toward the formation of an inorganic network containing a liquid phase (gel), which can be dried to remove the liquid phase from the gel thus forming a porous material. In contrast, the gelling process of the present invention does not involve hydrolysis or polycondensation reactions, and it forms a network of water-insoluble polymeric chains dispersed in the polar organic solvent.

The gelled mixture as described hereinabove can be subjected to de-gassing, in which the gelled mixture is placed under a reduced pressure or vacuum conditions, so as to remove the expandable fluid from the gelled mixture. Subsequently, a second solvent that is immiscible with the polar organic solvent previously used for swelling/gelling the microspheres can be added into the de-gassed gelled mixture with sufficient agitation, so as to “quench” the gelled mixture by separating the swelled microspheres from one another. For example, when the polar organic solvent is acetone, the second solvent can be water, which is immiscible with acetone. Due to the immiscibility between the polar organic solvent and the second solvent, the microspheres become more spatially separated from one another and therefore more dispersed. Such further dispersion of the microspheres functions to minimize the risk of coalescence during subsequent drying of the gelled mixture. Further separation of the microspheres can be achieved by a filtration or centrifugation step, which is optional for the purpose of the present invention.

After the de-gassing and quenching steps, both the polar organic solvent and the second solvent are preferably removed from the gelled mixture to form dry, free-flowing powders containing the microspheres with the solid particles entrapped therein. Removal of the polar organic solvent and the second solvent can be readily achieved by various separation and/or drying techniques well known in the art, such as decantation, centrifugation, filtration, solvent extraction, air drying, vacuum drying, freeze drying, spray drying, fluid bed drying, supercritical fluid drying, and the like. The polymeric shells, which have been previously swelled by the polar organic solvent and become porous with micro-channels extending therethrough, shrink significantly and lose their porosity after being dried. In other words, the micro-channels formed through the swelled polymeric shells of the microspheres during the gelling step close up after the drying step, thereby securely entrapping the pigment particles inside the microspheres. To minimize agglomeration between the dried microspheres, the resulting powders can be further subject to milling and sieving through one or more screens.

In order to eliminate or minimize the potential risk of the entrapped pigment particles leaking out of the dried microspheres, the resulting dry, free-flowing powders can be coated or otherwise surface-treated with a film-forming material, which forms a liquid-impermeable membrane over each of the dried microspheres. In this manner, the dried microspheres are sealed from solvents in the surrounding environment, which may potentially re-swell the polymeric shells of the microspheres and cause the entrapped pigment particles to leak out.

Any material capable of forming a liquid-impermeable membrane, either hydrophilic or hydrophobic, can be used in the present invention. Suitable materials include film-forming materials such as natural or synthetic homo- or co-polymers comprised of ethylenically unsaturated monomers including acrylic acid, methacrylic acid or their C₁-C₁₀ alkyl esters, ethylene, propylene, or vinylpyrrolidones; silicone gums, which are organosiloxanes generally having a viscosity ranging from about 200,000 to 10,000,000 centipoise at room temperature; animal, vegetable, silicone or mineral waxes; organic ester or hydrocarbon oils, or silicone resins such as trimethylsiloxy silicate or polymethylsilsesquioxane; cellulosic polymers; fatty acids (e.g. fatty carboxylic acids having from about 6 to 40 carbon atoms that may be liquid, solid or semi-solids at room temperature), fatty alcohols (e.g. alcohols having from 6 to 50 carbon atoms that may be liquid, solid, or semi-solid at room temperature), and inorganic materials. Preferably, but not necessarily, the film-forming material comprises an alkyl silicone polymer or more specifically a fatty alkylmethylsiloxane, such as cetyl dimethicone, stearyl dimethicone, or behenyl dimethicone, or other modified siloxanes, such as polyoxyalkylenated silicones typically referred to as dimethicone copolyol or cetyl dimethicone copolyol. For example, a polymethylhydrogensiloxane, which is commercially available from Dow Corning Corporation at Midland, Mich. under the trade name of Dow Corning® MH 1107 fluid, can be used as the film-forming material in the present invention. This polymethylhydrogensiloxane material is a colorless silicone liquid that can be heat cured in the presence of a catalyst (e.g., zinc octoate, iron octoate, dibutyl tin dilaurate, and tin octoate) to form a solid, liquid-impermeable membrane comprised of cross-linked dimethicone over the microspheres of the present invention. For another example, silicone copolymers commercialized by Dow Corning under the trade name of BIO-PSA, which are formed by reacting a siloxane resin with a diorganosiloxane, can also be used as film-forming materials in the present invention to form the liquid-impermeable membrane over the microspheres. Among various types of BIO-PSA materials available from Dow Corning, the Dow Corning® 7-4404, 7-4405, and 7-4411 fluids (containing trimethylated silica treated with dimethylsiloxane and dispersed in a cosmetically acceptable solvent, such as octamethyltrisiloxane, isododecane, or decamethyltetrasiloxane) are particularly preferred.

The resulting microspheres with the pigment particles entrapped therein and the liquid-impermeable membrane coated thereover may have an average particle size ranging from about 1 to about 50 microns, more preferably from about 1 to about 15 microns, and most preferably from about 5 to about 8 microns, as determined by a Malvern Particle Size Analyzer, available from Malvern Instrument at Worcestershire, UK. The entrapped pigment particles may account for from about 10% to about 90% of the total weight of the resulting microspheres, more preferably 30% to about 75% of the total weight, and most preferably from about 40% to about 60% of the total weight. The polymeric shells may account for from about 5% to about 75% of the total weight of the resulting microspheres, more preferably from about 10% to about 60% of the total weight, and most preferably from about 30% to about 50% of the total weight. The liquid-impermeable coating material may account for from about 1% to about 30% of the total weight of the resulting microspheres, more preferably from about 5% to about 20% of the total weight, and most preferably from about 10% to about 15% of the total weight.

The figure illustratively shows schematic views of an untreated hollow microsphere 10 and a microsphere 20 according to one embodiment of the present invention, which is formed by processing the untreated hollow microsphere 10 according to the method described hereinabove. Specifically, the untreated hollow microsphere 10 includes a gas-tight and deformable polymeric shell 12 with an expandable fluid 14 entrapped therein. The diameter of the untreated hollow microsphere 10 is approximately 20 microns. In contrast, the microsphere 20 of the present invention includes a collapsed polymeric shell 22 with pigment particles 24 entrapped therein and a liquid-impermeable membrane 24 coated thereover. The diameter of the microsphere 20 is significantly smaller than that of the untreated hollow microsphere 10 and approximately ranges from about 5 to about 8 microns.

When formulated into topical compositions, the microsphere-entrapped pigment particles of the present invention provide various advantages and benefits that are not available in their un-encapsulated or “naked” counterparts.

The most surprising and unexpected advantage is the significantly enhanced color intensity of the microsphere-entrapped pigment particles in comparison with the un-encapsulated or “naked” pigment particles.

Further, because the entrapped pigment particles are sealed off from potentially destabilizing or degrading active ingredients in the topical composition, they are significantly more stable than their un-encapsulated or “naked” counterparts. Entrapment by microspheres may also alter the hydrophobilicity or hydrophilicity of the pigment particles and allow such pigment particles to be formulated into aqueous, oil or silicone phases that are typically incompatible with un-encapsulated or “naked” pigment particles.

Because the microspheres of the present invention are formed by entrapping pigment particles in pre-formed, hollow polymeric microspheres that are subsequently collapsed during the entrapment process, rather than conventional in situ formation of polymeric coatings or matrixes around the pigment particles, the microspheres of the present invention are characterized by substantially more uniform particle sizes and reduced agglomeration between the microspheres. Further, the entrapment process of the present invention allows the pigment particles to be entrapped into microspheres that are many times larger in size than the pigment particles themselves (e.g., 10×, 20×, 50×, or 100×) within a relatively short period of time, while the conventional in situ coating or matrix-forming process is very time-consuming and can only form microspheres of limited sizes.

The microsphere-entrapped pigment particles of the present invention can be added directly to any pharmaceutically or cosmetically acceptable carrier to form a cosmetic or topical composition. For purpose of the present invention, pharmaceutically or cosmetically acceptable carriers are substances that are biologically compatible with human skin and can be used to formulate active ingredients described hereinabove and/or hereinafter into a cream, gel, emulsion, liquid, suspension, powder, nail coating, skin oil, or lotion that can be topically applied. In the case where the cosmetically acceptable carrier is in the form of an emulsion, it may contain from about 0.1 to 99%, preferably from about 0.5 to 95%, more preferably from about 1 to 80% by weight of the total composition of water and from about 0.1 to 99%, preferably from about 0.1 to 80%, more preferably from about 0.5 to 75% by weight of the total composition of oil. In the case where the composition is anhydrous it may comprise from about 0.1 to 90 wt % of oil and from about 0.1 to 75 wt % of other ingredients such as pigments, powders, non-aqueous solvents (such as mono-, di-, or polyhydric alcohols, etc. In the case where the composition is in the form of an aqueous based gel, solution, or suspension, it may comprise from about 0.1 to 99 wt % of water and from about 0.1 to 75 wt % of other ingredients such as botanicals, non-aqueous solvents, etc.

Suitable components of the pharmaceutically or cosmetically acceptable carrier include, but are not limited to: moisturizing agents, astringent agents, chelating agents, sequestrants, emulsifiers/surfactants, emollients, preservatives, stabilizers, abrasives, adsorbents, thickeners, gellants, solidifying/structuring agents, anti-caking agents, anti-foaming agents, pH buffering/adjusting agents, binders, film formers, humectants, pigments, opacifiers, essential oils, fragrances, and aromatic compounds. The pharmaceutically or cosmetically acceptable carrier or carriers can be present in the topical or cosmetic composition of the present invention at an amount ranging from about 1% to about 99.9%, preferably from about 50% to about 99.5%, more preferably from about 70% to about 99%, and most preferably from about 80% to 90% by total weight of the topical or cosmetic composition.

The topical or cosmetic composition may contain one or more skin care additives, which are agents that provide benefits to the skin, rather than merely improving the physical or aesthetic characteristics of the topical composition. If present, such skin care actives may range from about 0.01 to 50%, preferably from about 0.05 to 35% by weight of the total composition. Exemplary skin care additives that can be used in the topical or cosmetic compositions of the present invention include, but are not limited to: sunscreen agents, self-tanning agents, anti-aging agents, anti-wrinkle agents, anti-acne agents (e.g., resorcinol, salicylic acid, and the like), enzyme-inhibiting agents, collagen-stimulating agents, agents for the eradication of age spots and keratoses, analgesics, anesthetics, antimicrobials (e.g., antibacterials, antiyeast agents, antifungal agents, and antiviral agents), antidandruff agents, antidermatitis agents, antipruritic agents, antiemetics, anti-inflammatory agents, antihyperkeratolytic agents, antiperspirants, antipsoriatic agents, antiseborrheic agents, antihistamine agents, skin lightening agents, depigmenting agents, skin soothing/healing agents (e.g., aloe vera extract, allantoin, and the like), corticosteroids, hormones, antioxidants, proteins or peptides, vitamins and derivatives thereof (e.g., vitamin A, vitamin E, vitamin B₃, vitamin B₅, and the like), exfoliants, retinoids (e.g., retinoic acid and retinol), farnesol, bisabolol, phytantriol, glycerol, urea, guanidine (e.g., amino guanidine), clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, minocycline, hydroquinone, naproxen, ibuprofen, theophylline, cromolyn, albuterol, topical steroids (e.g., hydrocortisone, hydrocortisone 21-acetate, hydrocortisone 17-valerate, and hydrocortisone 17-butyrate), betamethasone valerate, betamethasone diproprionate, benzoyl peroxide, crotamiton, propranolol, promethazine, and mixtures or derivatives thereof. In a preferred, but not necessary embodiment of the present invention, the topical composition comprises one or more skin care actives selected from the group consisting of sunscreen agents, self-tanning agents, anti-aging agents, anti-wrinkle agents, anti-acne agents, antimicrobials, anti-inflammatory agents, skin-lightening agents, antioxidants, proteins or peptides, vitamins and derivatives thereof, exfoliants, and mixtures thereof.

For example, the topical or cosmetic compositions of the present invention may include one or more antioxidants, and more preferably one or more water-soluble extracts of biological materials that exhibit anti-oxidant activities. If present, such antioxidants or water-soluble extracts with anti-oxidant activities may range from about 0.01 to 45%, preferably from about 0.05 to 20%, more preferably from about 0.1 to 15% by weight of the total composition. Examples of suitable water-soluble extracts that exhibit anti-oxidant activities include, but are not limited to, extracts from: artemia, phytosphingosine, polygonum cuspidatum root, yeast such as saccharomyces lysate, thermos thermophillus ferment, birch (Betula alba), mimosa tenuiflora (bark) extract, fruit, clove, rye, malt, corn, spelt, millet, barley, oat, wheat, sesame, cumin, turmeric, green onion, celery, ginseng, ginger, licorice, carrot, bupleurum root, Ginkgo biloba (gingko), Foeniculi Fructus (fennel), kiwi, berry such as Moms bombycis (mulberry), Gentiana lutea (gentian), algae such as red algae, Arctium lappa (burdock), Salvia officinalis (sage), Lentinus edodes (shiitake mushroom), Perilla frutescens (perilla), Filipendula Multijuga, Fucus vesiculosis (bladderwrack, sea weed), peach kernel, Allium sativum (garlic), Poria cocos (poria), Humulus lupulus (hops), Mutan Cortex (Moutan Bark), Pimpinella major, Lactuca sative (lettuce), Astragalus membranaceous (astragalus) and Rosmarinus officinalis (rosemary), Prunus amygdalus (almond), Althea officinale (althea), aloe, Rosae Fructus (rose fruit, or Rosa multiflora), Scuttelaria baicalensis (Huang qin), Puerariae Radix (Pueraria Root, or Pueraria lobata), chamomile such as Chamomillae Flos (German chamomile), Gardenia jasminoides (zhii zi, Gardeniae Fructus), Sophora flavescens Aiton (Sophorae Radix), chlorella, rice bran, Paeoniae lactiflora (white peony), ziyu (Sanguisorba officinalis, burnet), Moms alba (sang bai pi, mulberry), Glycine max (soybean), Camellia sinensis (tea), Carthami Flos (safflower), Aesculus hippocastanum (horse chestnut), Melissa officinalis (lemon balm) and Coicis Semen (Coix lacryma-jobi var. ma-yuen), Angelica keisukei, Arnica montana (arnica), Foeniculum officinale (fennel), Isodon japonicus Hara (Isodonis Herba), Daucus Carota (carrot), Oryza sativa (rice), Crataegus cuneata (Japanese howthorn), Acores calamus (sweet flag), Crataegus oxycantha (howthorn), Juniperus communis, Ligusticum wallichii (Chinese lovage), Swertiae Herba (Swertia Herb), Thymus vulgaris (garden thyme), Citrus reticulata (Citrus unshiu), Capsicum tincture, Angelicae sinensis (angelica), Aurantii Pericarpium (bitter orange peel), Ruscus aculeatus (butcher's bloom), Vitis vinifera (grape), Tilia japonica (lime), Citrus junos and Rosa canina (rose hip), caffeine, Cinnamomi Cortex (cinnamon bark) and Eriobotrya japonica Lindl. (loquat), Gambir, Echinacea, Phellodendri Cortex (amur cork tree or Phellodendron amurense), Hypericum perforatum (St. John's wort), Citrus sinensis (orange), Valeriana fauriei Briquet, Artemisia capillaris Thunb., Cucumis sativus (cucumber), Geranii Herba (Geranium Herb), Lithospermum erythrorhizon Sieb. et Zucc., Hedera helix, Achillea millefolium (yarrow), Ziziphus jujuba (Chinese dates), Calendula officinalis (pot marigold), Houttuynia cordata (Houttuyniae Herba, Houttuynia Herba), Potentilla erecta, Petroselinum crispum (parsley), Parietaria officinalis, Santalum album (sandalwood), Prunus persica (peach), Centaurea cyanus (cornflower), Eucalyptus globulus (eucalyptus) and Lavandula angustifolia (lavender), Persea americana (avocado), Nasturtium officinalis (watercress), Symphytum officinale (comfrey), Asarum sieboldii (wild ginger), Xanthoxyum piperitum (Japan pepper), Rehmannia glutinosa (di huang), Mentha piperita (peppermint), Syzygium aromaticum (clove), Tussilago farfara (coltsfoot) and Haematoxylum campechianum (logwood); Oolong tea, Cinchona succirubra (peruvian bark), Betula verrucosa (birch) and Glechoma hederacea (ground ivy), milk and royal jelly, honey, cysteine and derivatives thereof, ascorbic acid and derivatives thereof, BHA, BHT, ferulic acid and derivatives thereof, grapeseed extract, pine bark extract, horseradish extract, hydroquinones, rosmarinic acid, coffee robusta seed, caffeic acid, tocopherol and derivatives thereof, green tea extract, sodium DNA, sodium ribonucleic acid, octyl, propyl and dodecyl gallates, uric acid and thiodiproprionate derivatives.

In a preferred, but not necessary, embodiment of the present invention, one or more of the antioxidant agents as listed hereinabove are co-entrapped into the microspheres together with the pigment particles of the present invention. Such co-entrapment can be achieved, for example, by mixing such antioxidant agents together with the pigment particles, the hollow microspheres, and the polar organic solvent during the gelling step to form the gelled mixture. Antioxidant agents particularly preferred for co-entrapment with the pigment particles of the present invention include, for example, tetrahydrocurcuminoids, ascorbyl tocopheryl maleate (also referred to as 2-CME), grape seed extract, and rosemary extract. A blend or mixture containing all of these particularly preferred antioxidant agents in equal or substantially equal quantities is most preferred for the practice of the present invention. Alternatively, the antioxidant agents can be used to form an antioxidant coating over the microspheres. Further, the antioxidant agents can be provided in a solubilized or dispersed form in the cosmetically or pharmaceutically acceptable carrier of the topical or cosmetic compositions of the present application. Such co-entrapped, coated or dispersed antioxidant agents function to scavenge or neutralize free oxygen radicals from various sources in the topical or cosmetic compositions, thereby further improving the overall stability of the topical or cosmetic compositions of the present invention.

The cosmetic or topical composition of the present invention may also comprise one or more inorganic or organic sunscreen agents to protect the skin against potential damages by UV radiation. Exemplary inorganic sunscreen agents include, but are not limited to: titanium dioxide, zinc oxide, and the like. Exemplary organic sunscreen agents include, but are not limited to: benzophenones and derivatives thereof (e.g., benzophenone-3, dioxybenzone, sulisobenzone, octabenzone, hydroxy- and/or methoxy-substituted benzophenones, and benzophenonesulfonic acids and salts thereof); salicylic acid derivatives (e.g., ethylene glycol salicylate, triethanolamine salicylate, octyl salicylate, homomethyl salicylate, and phenyl salicylate); urocanic acid and derivatives thereof (e.g., ethyl urocanate); p-aminobenzoic acid (PABA) and derivatives thereof (e.g., ethyl/isobutyl/glyceryl esters thereof and 2-ethylhexyl p-dimethylaminobenzoate, which is also referred to as octyldimethyl PABA); anthranilates and derivatives thereof (e.g., o-amino-benzoates and various esters of amino-benzoic acid); benzalmalonate derivatives; benzimidazole derivatives; imidazolines; bis-benzazolyl derivatives; dibenzoylmethanes and derivatives thereof (e.g., 4-tert-butyl-4′-methoxydibenzoylmethane, which is commonly referred to as “avobenzone,” and 4-isopropyl-dibenzoylmethane); benzoazole/benzodiazole/benzotriazoles and derivatives thereof (e.g., 2-(2-hydroxy-5-methylphenyl)benzotriazole and methylene bis-benzotriazolyl tetramethylbutylphenol, which is commonly referred to as “Tinosorb M”); diphenylacrylates and derivatives thereof (e.g., 2-ethylhexyl-2-cyano-3,3-diphenylacrylate, which is commonly referred to as “octocrylene,” ethyl-2-cyano-3,3-diphenylacrylate, which is commonly referred to as “etocrylene,” and 2-ethylhexyl-2-cyano-3-(4′-methoxyphenyl)-3-phenylacrylate, which is commonly referred to as “methoxycrylene”); diesters or polyesters containing diphenylmethylene or 9H-fluorene substitutional groups; 2-phenyl-benzimidazole-5-sulphonic acid (PBSA); 4,4-diarylbutadienes; cinnamates and derivatives thereof (e.g., 2-ethylhexyl-p-methoxycinnamate, octyl-p-methoxycinnamate, umbelliferone, methylumbelliferone, methylaceto-umbelliferone, esculetin, methylesculetin, and daphnetin); camphors and derivatives thereof (e.g., 3-benzylidenecamphor, 4-methylbenzylidenecamphor, polyacrylamidomethyl benzylidenecamphor, benzylidene camphor sulfonic acid, and terephthalylidene dicamphor sulfonic acid, which is commonly referred to as “Encamsule”); triazines and derivatives thereof (e.g., 2,4-bis-{[4-(2-ethyl-hexyloxy)-2-hydroxy]-phenyl}-6-(4-methoxyphenyl)-1,3,5-triazine, which is commonly referred to as “Tinosorb S”); naphthalates and derivatives thereof (e.g., diethylhexyl-2,6-naphthalate); naphtholsulfonates and derivatives thereof (e.g., sodium salts of 2-naphthol-3,6-disulfonic and 2-naphthol-6,8-disulfonic acids); dibenzalacetone and benzalacetonephenone; diphenylbutadienes and derivatives thereof; di-hydroxynaphthoic acid and salts thereof; o- and p-hydroxybiphenyldisulfonates; coumarin derivatives (e.g., 7-hydroxy, 7-methyl, and 3-phenyl derivatives thereof); azoles/diazoles/triazoles and derivatives thereof (e.g., 2-acetyl-3-bromoindazole, phenyl benzoxazole, methyl naphthoxazole, and various aryl benzotriazoles); quinine and derivatives thereof (e.g., bisulfate, sulfate, chloride, oleate, and tannate salts thereof); quinoline and derivatives thereof (e.g., 2-phenylquinoline and 8-hydroxyquinoline salts); tannic acid and derivatives thereof (e.g., hexaethylether derivatives thereof); hydroquinone and derivatives thereof; uric acid and derivatives thereof; vilouric acid and derivatives thereof, and mixtures or combinations thereof. Salts and otherwise neutralized forms of certain acidic sunscreens from the list hereinabove are also useful herein. These organic sunscreen agents may be used alone or in combination of two or more. In addition, other known animal or vegetable extracts having UV light-absorbing ability may properly be used alone or in combination.

Organic sunscreen agents that are particularly useful for the practice of the present invention are: 4,4′-t-butyl methoxydibenzoylmethane, 2-ethylhexyl-2-cyano-3,3-diphenylacrylate, 2-ethylhexyl-2-cyano-3-(4′-methoxyphenyl)-3-phenylacrylate, 2-ethylhexylsalicylate, 3,3,5-trimethylcyclohexylsalicylate, 2-ethylhexyl p-methoxycinnamate, 2-hydroxy-4-methoxybenzophenone, 2,2-dihydroxy-4-methoxybenzophenone, 2,4-bis-{4-(2-ethyl-hexyloxy)-2-hydroxyl-phenyl}-6-(4-methoxyphenyl)-1,3,5-triazine, methylene bis-benzotriazolyl tetramethylbutylphenol, terephthalylidene dicamphor sulfonic acid, diethylhexyl 2,6-naphthalate, digalloyltrioleate, ethyl 4-[bis(hydroxypropyl)]aminobenzoate, glycerol p-aminobenzoate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid, and mixtures or combinations thereof. More preferably, the sunscreen compositions of the present invention further include a second organic sunscreen agent selected from the lists provided hereinabove.

The above-described sunscreen agents may be solubilized or freely dispersed in the cosmetically or pharmaceutically acceptable carrier of the topical or cosmetic compositions of the present application. Alternatively, the sunscreen agents can be provided in a protected form, i.e., encapsulated in protective structures. For example, the sunscreen agents can also be encapsulated or entrapped into microspheres similar to those described hereinabove, i.e., with collapsed polymeric shells.

The cosmetically acceptable carrier may also contain one or more oils, which may be silicone, organic, or mixtures thereof. If present, such oils may range from about 0.1 to 99% by weight of the total composition and include volatile or non-volatile silicones such as cyclomethicone; methyl trimethicone; octamethyltrisiloxane; decamethyltetrasiloxane; dodecamethylpentasiloxane; dimethicone; phenyl trimethicone trimethylsiloxyphenyl dimethicone; phenyl dimethicone; cetyl dimethicone; dimethicone copolyol, cetyl dimethicone copolyol; glycerolated silicones such as lauryl PEG-9 polydimethylsiloxyethyl dimethicone; or mixtures thereof. Suitable esters include mono-, di-, or triesters of C4-30 fatty acids and mono-, di-, or polyhydric C1-20 alcohols, such as fatty acid (e.g., stearyl, behenyl, and isostearyl) esters of glycerin, or fatty acid esters of alpha hydroxyl acids such as citric, malic, or lactic acids and the like. Suitable hydrocarbons include monomeric or polymeric olefins or alpha olefins, such as polyisobutene, polydecene, polybutene, or hydrogenated derivatives thereof.

The cosmetically acceptable carrier may also comprise one or more humectants. If present, they may range from about 0.1 to 20% by weight of the total composition and include C₁₋₄ alkylene glycols such as butylene, propylene, ethylene glycol, glycerin and the like.

The cosmetically acceptable carrier may also contain one or more waxes preferably having a melting point ranging from about 30 to 150° C. If present, such waxes may range from about 0.1 to 45% by weight of the total composition and include animal, vegetable, mineral, or silicone waxes. Examples include alkyl dimethicones stearyl dimethicone, candelilla, polyethylene, ozokerite, beeswax, and the like.

The cosmetically acceptable carrier may also comprise one or more organosiloxane elastomers, either emulsifying or non-emulsifying. If present, such elastomers may range from about 0.1 to 30% by weight of the total composition. Examples of suitable elastomers include dimethicone/vinyl dimethicone crosspolymer; dimethicone/dimethicone PEG/PPG 10/15 crosspolymer; and the like.

The cosmetically acceptable carrier may also comprise one or more nonionic surfactants, particularly if the topical or cosmetic composition of the present invention is provided in the emulsion form. If present, such surfactants may range from about 0.1 to 20% by weight of the total composition. Suitable surfactants include ethoxylated fatty C₆₋₃₀ alcohols such as steareth, beheneth, ceteth where the number following each of the surfactants refers to the number of repeating ethylene oxide groups which may range from 2 to 250, e.g. steareth-2, beheth-30 and so on.

While the present invention has been described hereinabove with reference to specific embodiments, features and aspects, it will be recognized that the invention is not thus limited, but rather extends in utility to other modifications, variations, applications, and embodiments, and accordingly all such other modifications, variations, applications, and embodiments are to be regarded as being within the spirit and scope of the present invention.

What we claim is:

1. A topical composition comprising a dispersion of microspheres in a cosmetically or pharmaceutically acceptable carrier, wherein each of said microspheres comprises a collapsed polymeric shell having entrapped therein one or more pigment particles.

2. The composition of claim 1, wherein the microspheres have an average particle size ranging from about 1 micron to about 50 microns, and the pigment particles have an average particle size ranging from about 0.001 micron to about 0.5 micron.

3. The composition of claim 1, wherein the collapsed polymeric shell comprises at least one synthetic polymer obtained by polymerization of one or more ethylenically unsaturated monomers selected from the group consisting of vinylidene chloride, vinyl chloride, acrylonitrile, acrylic acid and its C₁-C₂₀ aliphatic or aromatic esters, methacrylic acid and its C₁-C₂₀ aliphatic or aromatic esters, acrylamide, methacrylamide, vinyl pyrrolidone, alkenes, styrene, ethylene, propylene, butylene, methylpentene, and 1,3-butadiene.

4. The composition of claim 1, wherein the collapsed polymeric shell comprises at least one synthetic thermoplastic polymer selected from the group consisting of polyesters, polyamides, polyphthalamides, polyimides, polycarbonates, polyketones, cellulose acetate, polysulfones, polyphenylene sulfides, polyphenylene oxides, polylactic acids, polyvinylpyrrolidone, polystyrene, polyacrylonitrile, polyacrylamide, polymethylmethacrylate, polyacrylates, and copolymers thereof.

5. The composition of claim 1, wherein the collapsed polymeric shell comprises a copolymer of vinylidene chloride, acrylonitrile, and/or methyl methacrylate.

6. The composition of claim 1, wherein the entrapped pigment particles comprise colored iron oxides.

7. The composition of claim 1, wherein the entrapped pigment particles are characterized by an increase in color intensity compared with un-entrapped pigment particles.

8. The composition of claim 1, wherein each of said microspheres comprises two or more different types of pigment particles co-entrapped therein.

9. The composition of claim 1, wherein two or more microspheres contain two or more different types of pigment particles separately entrapped therein.

10. The composition of claim 1, wherein the collapsed polymeric shell is further coated with a liquid-impermeable membrane.

11. The composition of claim 10, wherein the liquid-impermeable membrane comprises one or more materials selected from the group consisting of acrylate homo- or co-polymers, methacrylate homo- or co-polymers, vinylpyrrolidone homo- or co-polymers, silicone gums, silicone waxes, silicone oils, silicone resins, esters, hydrocarbons, celluloses, fatty acids, fatty alcohols, and inorganic materials.

12. The composition of claim 10, wherein the liquid-impermeable membrane comprises crosslinked dimethicone or trimethylated silica treated with dimethyl siloxane.

13. The composition of claim 1, further comprising one or more antioxidants.

14. The composition of claim 13, wherein said one or more antioxidants are co-entrapped with the pigment particles inside the collapsed polymeric shell of each microsphere, or coated over the microspheres, or solubilized or dispersed in the cosmetically or pharmaceutically acceptable carrier.

15. A microsphere comprising a collapsed polymeric shell having entrapped therein one or more pigment particles, wherein the collapsed polymeric shell is further coated with a liquid-impermeable membrane.

16. A method for modifying or treating pigment particles, comprising:
(a) forming a gelled mixture by mixing either simultaneously or sequentially in any order (1) hollow microspheres each comprising a deformable polymeric shell having entrapped therein an expandable fluid, (2) a polar organic solvent capable of swelling but not dissolving the polymeric shells of the hollow microspheres, and (3) pigment particles, wherein micro-channels are formed in the swelled polymer shells to allow entry of the pigment particles into the hollow microspheres and exit of the expandable fluid therefrom, thereby forming microspheres that each comprises a collapsed polymeric shell in a gelled state and has one or more of said pigment particles entrapped therein;
(b) removing the expandable fluid and the polar organic solvent from the gelled mixture; and
(c) coating the microspheres with a film-forming material to form a liquid-impermeable membrane thereon.

17. The method of claim 16, wherein the polar organic solvent is selected from the group consisting of dimethylformamide, dimethylchloride, trichloroethylene, chloroform, methanol, ethanol, isopropanol, acetone, ethyl acetate, butyl acetate, and methyl ethyl ketone.

18. The method of claim 16, wherein the expandable fluid is selected from the group consisting of gases, air, nitrogen, volatile liquid hydrocarbons, isobutane, and isopentane.

19. The method of claim 16, wherein the expandable fluid is first removed from the gelled mixture by de-gassing at a reduced pressure or under vacuum conditions, followed by a quenching step in which water is added to the gelled mixture to allow separation of the microspheres from one another before removal of the polar organic solvent.
